(19) United States
(12) Patent Application Publication
(10) Pub. No.: US 2023/0320643 A1
(43) Pub. Date: Oct. 12, 2023

(54) VR/AR PHOBIA TRAINING IN A CONTROLLED ENVIRONMENT WITH STRESS LEVEL SENSORS AND MANAGEMENT THROUGH SCENARIOS CONTROL

(71) Applicant: PsyTechVR, Frederick, MD (US)

(72) Inventors: Ivan ANDREEV, Frederick, MD (US); Peter OYKHMAN, Frederick, MD (US); Daniil ANDREEV, Frederick, MD (US)

(21) Appl. No.: 18/133,275

(22) Filed: Apr. 11, 2023

Related U.S. Application Data
(60) Provisional application No. 63/329,526, filed on Apr. 11, 2022.

(51) Int. Cl. A61B 5/16; A61B 5/024; A61B 5/369; A61B 5/00; G06F 3/01

(52) U.S. Cl. CPC A61B 5/165; A61B 5/024; A61B 5/369; A61B 5/0002; G06F 3/011; G06F 3/016

(57) ABSTRACT

Systems, devices, methods, or computer-readable instructions that provide an AR or VR video stream including a sequence of frames that simulate a phobia, and for each frame or subset of frames, monitoring and storing one or more biometrics of a user.

### PRIORITY INFORMATION

This application claims the benefit of U.S. Provisional Patent Application No. 63/329,526 filed on Apr. 11, 2022, which is hereby incorporated by reference in its entirety.

### FIELD OF THE INVENTION

The embodiments of the present invention generally relate to the use of one or more biometrics, and more particularly, to the use of one or more biometric sensors and/or sensor types in virtual reality (VR) or augmented reality (VR) controlled environments for phobia training.

### DISCUSSION OF THE RELATED ART

In general, biometrics may be used to track vital signs that provide indicators about a person's physical state, including stress level, and may be used in a variety of ways. As an example, for health monitoring, vital signs may be used to screen for health risks (e.g., temperature). While sensing temperature is a well-developed technology, collecting other useful and accurate vital signs such as heart rate and blood pressure can be achieved using appropriate sensors.

Phobias are widespread causes of stress. Common example phobias include arachnophobia (fear of spiders), ophidiophobia (fear of snakes), acrophobia (fear of heights), aerophobia (fear of flying), cynophobia (fear of dogs), astraphobia (fear of thunder and lightning), trypanophobia (fear of injections), etc. It is estimated that as many as 15 percent of people have one or more phobias.

Despite the prevalence of phobias, many people remain reluctant to seek treatment for a variety of reasons. Many people are not ready to share their fears with strangers, even health care professionals. People may minimize their phobias as they consider their fears insignificant, and try to cope with them individually. Some people believe, or hope, that their fears will clear up on their own, and without treatment. In terms of treatment to date, “Emotional Mental Imagery of the Situation” does not allow to “push” the patient against the object of his/her phobia while being in the office environment during therapy. It is not feasible to provide patients with effective real-life therapy. For example, taking an aerophobia patient (fear of flying) to a real airplane flight is correlated with unjustified risks, time, and financial costs.

Additionally, and from the patient's perspective, it takes significant money and time to periodically visit a health care professional, and the likelihood of a cure is low. Moreover, in response to the COVID pandemic, more and more people prefer to work from home and see health care professionals remotely. Remote living can result in the development of additional fears. Additionally, remote living can generate further challanges for productive offline communication with health care professionals.

Accordingly, the inventors have developed systems, devices, methods, and computer-readable instructions that enable accurate capture of one or more biometric indicators during a patient's near real life experience with phobia in a controlled AR/VR environment.

### SUMMARY OF THE INVENTION

Accordingly, the embodiments of the present invention are directed to VR/AR phobia training in a controlled environment with stress level sensors and management through scenarios control that substantially obviates one or more problems due to limitations and disadvantages of the related art.

Objects of the present invention provide systems, devices, methods, and computer-readable instructions that enable accurate capture of one or more biometric indicators during a patient's near real life experience with phobia in a controlled AR/VR environment.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the VR/AR phobia training in a controlled environment with stress level sensors and management through scenarios control includes systems, devices, methods, and computer-readable instructions for providing an AR or VR video stream including a sequence of frames that simulate a phobia, for each frame or subset of frames, monitoring and storing one or more biometrics of a user.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates a system according to an example embodiment of the present invention.

FIG. 2 illustrates a system according to another example embodiment of the present invention.

FIGS. 3A-3C illustrate patient user-interfaces according to example embodiments of the present invention.

FIG. 4 illustrates a healthcare professional user-interface according to an example embodiment of the present invention.

FIG. 5 illustrates VR/AR glasses 500 with a plurality of integrated sensors 1-6 according to the example embodiments.

FIG. 6 illustrates a flow diagram of functionality 600 for using the VR/AR simulated phobia according to an example embodiment of the present invention.

FIG. 7 illustrates a representative architecture of a portable electronic device 700 according to an example embodiment.

### DETAILED DESCRIPTION OF THE INVENTION

Reference will now be made in detail to the embodiments of the present invention, examples of which are illustrated in the accompanying drawings.

Embodiments of user interfaces and associated methods for using system and device are described. The user interface can include a touch screen, one or more wearable devices (e.g., AR/VR Glasses such as Oculus or Google Glasses), one or more handheld or wearable controllers (e.g., haptic-enabled controllers, haptic-enabled glove), a gyroscopic or other acceleration device, one or more biometric devices (e.g., heart rate detector, blood pressure monitor, a camera configured for pupil/iris biometric collection, a microphone configured for voice biometric collection, and/or other biometric devices). Other biometric measurements may include continuous blood oxygen, finger temperature, heart rate variability, single-lead electrocardiogram (ECG), electroencephalogram (EEG), and the like. The various biometrics enable the healthcare professional to track patient's baseline physiological state as well as the physiological state when the user is experiencing a phobia in AR/VR.

It should be understood, however, that the user interfaces and associated methods can be applied to other devices, such as a portable communication device such as a tablet or mobile phone. The portable communication device can support a variety of applications, such as wired or wireless communications. The various applications that can be executed on the device can use at least one common physical user-interface device, such as a touch screen. One or more functions of the touch screen as well as corresponding information displayed on the device can be adjusted and/or varied from one application to another and/or within a respective application. In this way, a common physical architecture of the device (e.g., processor, memory, communication bus) can support a variety of applications with user interfaces that are intuitive and transparent.

The embodiments of the present invention provide systems, devices, methods, and computer-readable instructions to measure one or more biometrics, such as heartrate, blood pressure, the aforementioned, and the like in response to an AR/VR generated phobia. In the various embodiments, the systems, devices, methods, and instructions collect, process, and analyze the biometric response to the AR/VR generated phobia. A patient's state (e.g., stressed, relaxed, anxious, etc.) and progress (e.g., in response to a simulated AR/VR phobia or meditation) may be evaluated by a healthcare professional (e.g., a psychiatrist or psychologist) using a variety of interfaces that track a patient's biometric information to better understand the patient's physiological state. By using one or more biometric indicators, the patient's physiological state is objectively assessed by the collected data rather than subjectively evaluated in response to the questions posed by the healthcare professional.

As will be readily understood, the embodiments may be configured for both in-person patient visits as well as remote tele-visits. In other words, the patient and healthcare professional may be in near proximity or the patient and healthcare professional also may be remote and communicatively connected over the Internet.

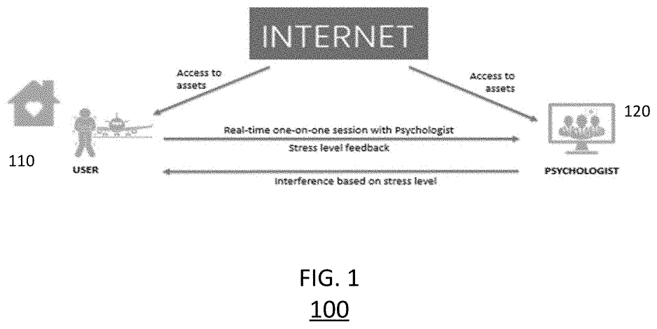

FIG. 1 illustrates a system 100 according to an example embodiment of the present invention.

As illustrated in FIG. 1, system 100 enables Internet-based tele-visits between patient 110 and heath care professional 120. Using one or more biometric measurements, heath care professional 120 may monitor the physiological state (e.g., increasing or decreasing stress level) of patient 110 in response to an AR/VR scenario.

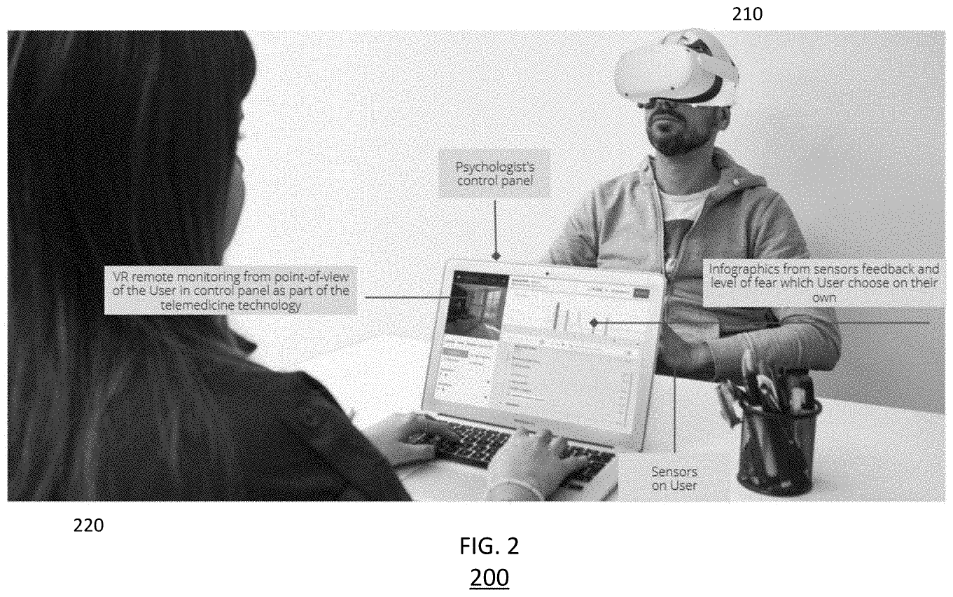

FIG. 2 illustrates a system 200 according to another example embodiment of the present invention.

As illustrated in FIG. 2, system 200 enables in-person visits between patient 210 and heath care professional 220. Using one or more biometric measurements, heath care professional 220 may locally monitor the physiological state (e.g., increasing or decreasing stress level) of a patient in response to an AR/VR scenario.

The various AR/VR scenarios are configured to simulate a variety of fears or phobias. Additionally, the various AR/VR scenarios are configured to simulate one or more triggers for an anger response (e.g., problem at work, problem at home, etc.). In another example, the various AR/VR scenarios are configured to simulate one or more triggers of a post-traumatic stress disorder (PTSD) response (e.g., various types of violence, bullying, weather event, fire, explosion, etc.). In another example, the various AR/VR scenarios are configured to simulate one or more events to induce calmness, such as a meditation scenario.

Accordingly, the embodiments of the present invention include one or more software-enabled applications for guided (e.g., self-guided or guided by a health care professional) trainings to enable a user to experience one or more fears and phobias in AR/VR. Additionally, the embodiments of the present invention include one or more interfaces for consultations by a healthcare professional, such as a psychiatrist, psychologist, psychotherapists, etc. to view the user in AR/VR, as well as his/her biometric indicators using stress measuring sensors (e.g., heart rate, blood pressure, etc.) and/or reported symptoms (e.g., headache, dizziness, sweatiness, etc.) with on-line real-time therapy/telemedicine.

As illustrated in FIGS. 1 and 2, the patients (or users) are provided with the following: 1. VR/AR glasses; 2. stress level sensors (e.g., one biometric devices, such as VR/AR glasses with a plurality of integrated sensors such as EEG, muscle contraction, heartrate, etc.); 3. access to the one or more AR/VR simulations (e.g., phobia or meditation) that are used to assess the user's physiological response to simulated phobia or meditation; and 4. access to schedule one-on-one telemedicine sessions with a healthcare professional. Additionally, the healthcare professionals are provided with 5. access in real-time to what the patient views (e.g., simulated phobias) in VR/AR glasses, 6. ability to monitor the patient's stress level through sensor data; and 7. ability to guide and if necessary interfere, interrupt, and/or adjust the patient experience in the simulation.

In the various embodiments, ARNR headsets provide a simulated phobia (e.g., viewing of a spider and/or a haptic generated sensation of spider crawling on hand or other body part) and biometric sensors that measure and store one or more biometric measurements that can be used to determine the user's level of stress. Alternatively, or additionally, the user may be provided with a mindfulness or relaxing simulation in some embodiments.

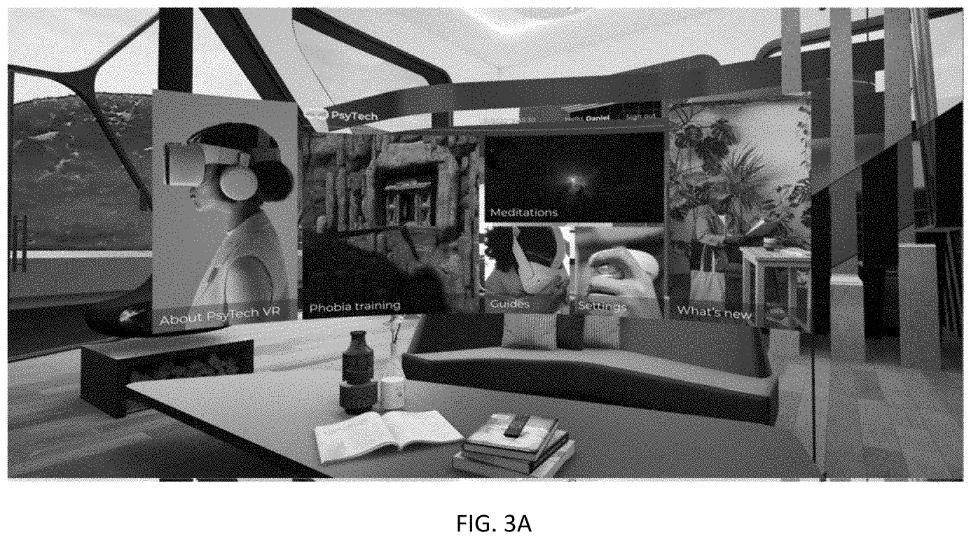

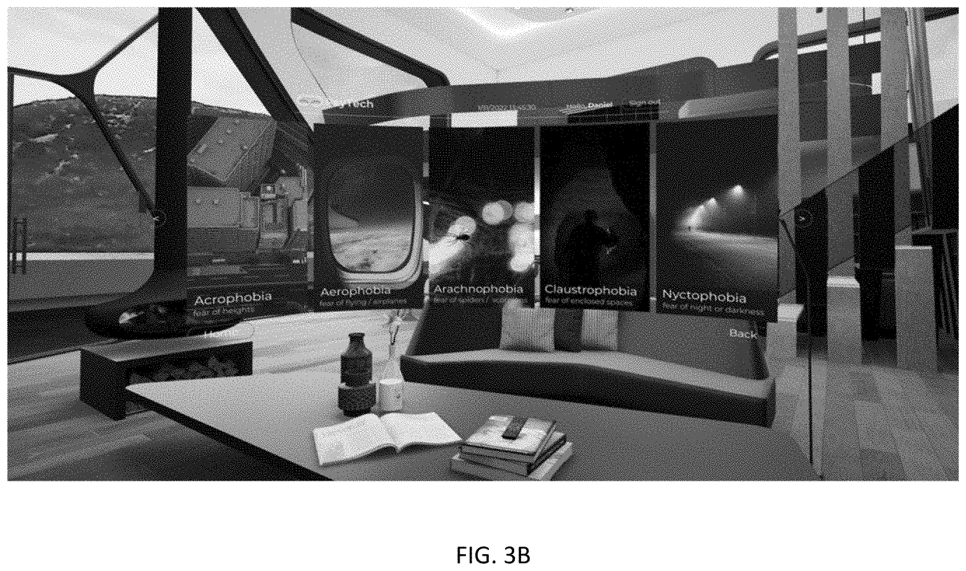

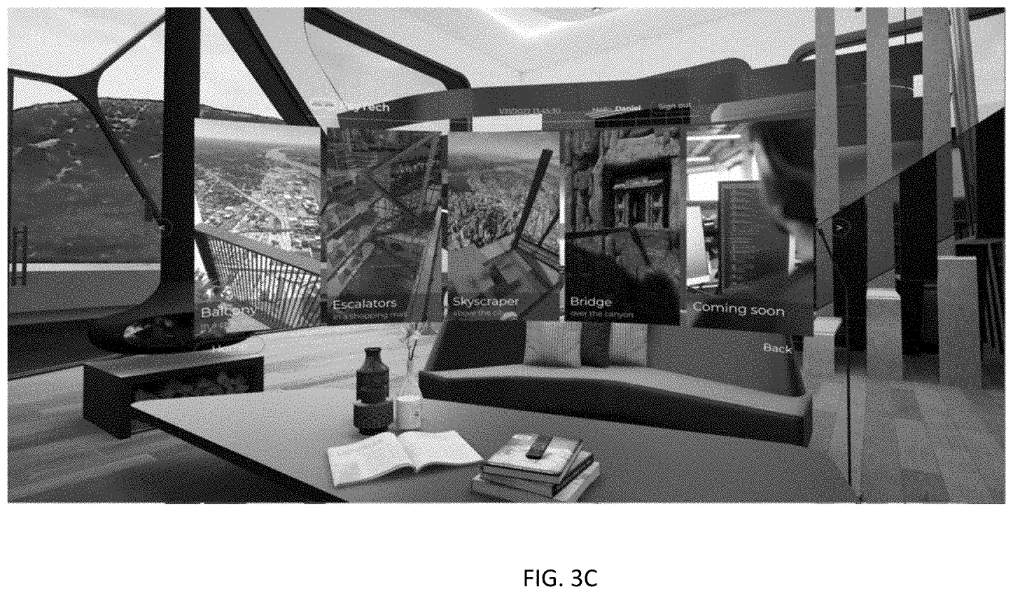

FIGS. 3A-3C illustrate patient user-interfaces according to example embodiments of the present invention. As illustrated in FIGS. 3A-3C, a patient may navigate through a variety of simulated phobias or meditations using ARNR glasses. In the example illustrated in FIG. 3C, the patient selects aerophobia, and may select from any of a balcony, escalator, skyscraper, or bridge to experience simulated aerophobia.

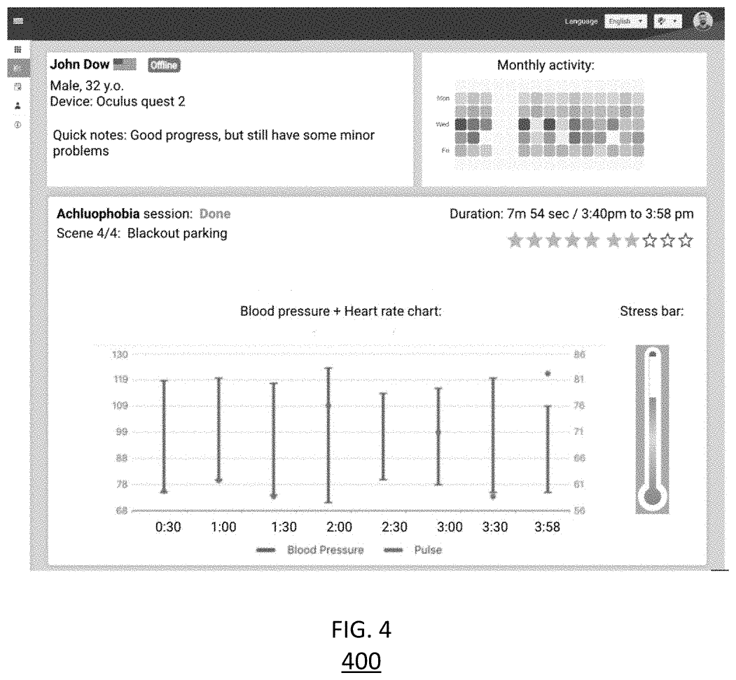

FIG. 4 illustrates a healthcare professional user-interface 400 according to an example embodiment of the present invention.

As illustrated in FIG. 4, the healthcare professional views the patient's heart rate and blood pressure at various time increments. In addition, the embodiments calculate the patient's stress level as illustrated in the stress bar. In general, stress is a state of psychological and physical tension in response to pathogens that have a provoking effect on the body. In the embodiments, patients experience fears and phobias, finding themselves in extreme situations in ARNR that are designed to expose the body to stress.

In this example embodiment, an additional feature for analytics is the stress level of the patient. This indicator is measured by heart rate in the following way: 1) the first heart rate (base figure) is measured before training, when the patient is calm; 2) average indicator of maximum heart rate is collected during the phobia simulation, getting indicators while facing fears and phobias; and 3) the last indicator is measured after the end of the phobia simulation. The stress bar compares the average maximum heart rate and the base heart rate.

Although heartrate and blood pressure are illustrated in FIG. 4, the embodiments may use a variety of biometric indicators such as pupil/iris biometric collection, a microphone configured for voice biometric collection, continuous blood oxygen, finger temperature, heart rate variability, electrocardiogram (ECG), electroencephalogram (EEG), and the like.

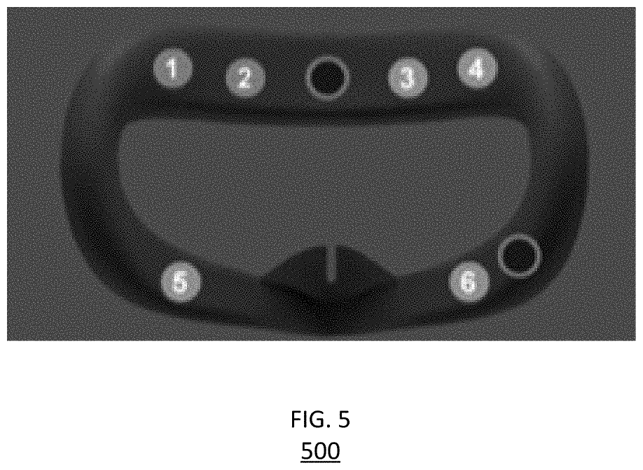

FIG. 5 illustrates VR/AR glasses 500 with a plurality of integrated sensors 1-6 according to the example embodiments. For example, the various sensors 1-6 may include EEG, muscle contraction, heartrate, etc.

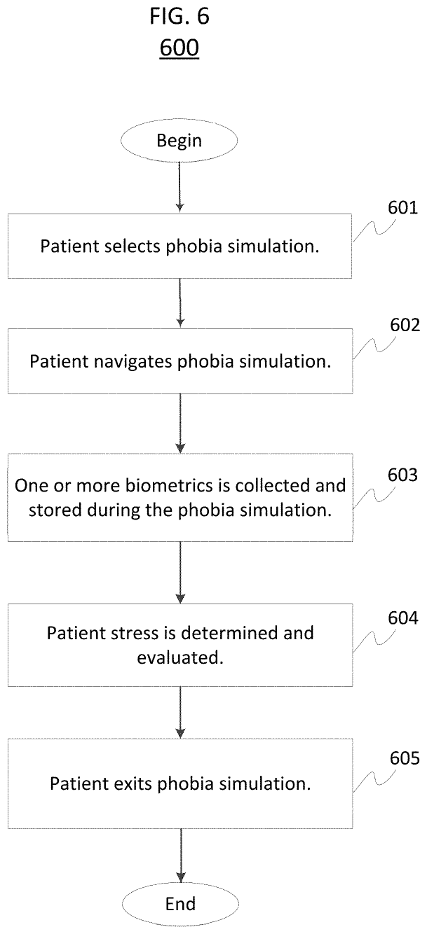

FIG. 6 illustrates a flow diagram of functionality 600 for using the VR/AR simulated phobia according to an example embodiment of the present invention. In some instances, the functionality of the flow diagram of FIG. 6 is implemented by software stored in memory or other computer-readable or tangible media, and executed by a processor. In other instances, the functionality may be performed by hardware (e.g., through the use of an application specific integrated circuit (“ASIC”), a programmable gate array (“PGA”), a field programmable gate array (“FPGA”), etc.), or any combination of hardware and software.

At the outset, functionality 600 may optionally initialize the components of the system (e.g., system 100 of FIG. 1 or system 200 of FIG. 2) such as the AR/VR glasses, the patient's computer that is communicatively coupled to the AR/VR glasses, as well as the computer of the heath care professional. At 601, the patient selects a phobia from a plurality of phobias for simulation. Next, at 602, the patient navigates the phobia simulation. For example, if the patient selects aerophobia, the patient may further select from any of a balcony, escalator, skyscraper, or bridge to experience simulated aerophobia. In the simulated phobia, an AR or VR video stream is provided to the patient/user including a sequence of frames that simulate a phobia. During the phobia simulation, one or more biometrics of the patient are collected at 603. The embodiments may use a variety of biometric indicators such as heart rate, blood pressure, pupil/iris biometric collection, a microphone configured for voice biometric collection, continuous blood oxygen, finger temperature, heart rate variability, electrocardiogram (ECG), electroencephalogram (EEG), and the like. For example, for each frame or subset of frames of the AR/VR video stream, one or more biometrics of the patient/user is monitored and stored. Based on the collected biometrics, the patient's physiological state and stress level are determined and evaluated by the health care professional, at 604. Lastly, the patient exits the phobia simulation at 605.

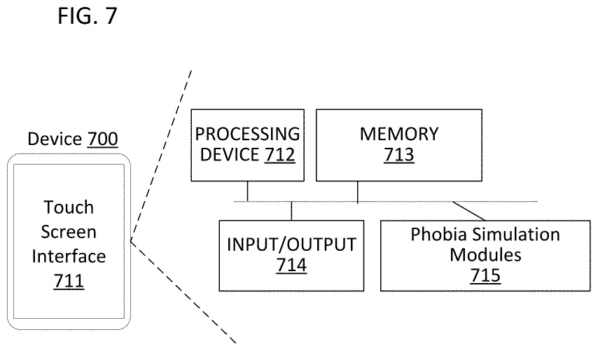

FIG. 7 illustrates a representative architecture of a portable electronic device 700 according to an example embodiment. The portable electronic device 700 may be communicatively coupled with one or more remote devices, such as AR/VR Glasses and the computer of the health care professional.

A portable electronic device 700 may include a touch screen interface 711, processing device 712, memory 713, and input/output modules 714. The touch screen interface 711 may include a display, which may be a touch screen, capable of displaying data to a user of the portable electronic device 700. Portable electronic device 700 may also include phobia simulation modules 715 that generally implement the functionality of the embodiments of the invention.

Although not shown, the touch screen may include a sensor that may be a capacitive touch detection sensor, configured to detect and track movement on the surface and/or in the vicinity of the display. The sensor may be coupled to a signal processing circuit that is configured to identify, locate, and/or track object movement based on the data obtained from sensor. The input/output module 714 manages the functionality of touch screen interfaced 711. For example, input/output module 714 may include functionality for identifying a component section within the personal information management application. An alternate component section may be selected by touching the alternate component section.

Memory 713 may include a computer readable medium storing application modules, which may include instructions associated with applications and modules of the portable electronic device 700.

The portable electronic device may contain a processing device 712, memory 713, and a communications device (not shown), all of which may be interconnected via a system bus. In various embodiments, the device 700 may have an architecture with modular hardware and/or software systems that include additional and/or different systems communicating through one or more networks via one or more communications devices.

Communications devices may enable connectivity between the processing devices 712 in the device 700 and other systems by encoding data to be sent from the processing device 712 to another system over a network and decoding data received from another system over the network for the processing device 712.

In an embodiment, memory 713 may contain different components for retrieving, presenting, changing, and saving data and may include computer readable media. Memory 713 may include a variety of memory devices, for example, Dynamic Random Access Memory (DRAM), Static RAM (SRAM), flash memory, cache memory, and other memory devices. Additionally, for example, memory 713 and processing device(s) 712 may be distributed across several different computers that collectively comprise a system. Memory 713 may be capable of storing user inputs and preferences as well as customized displays and templates. In some instances, a cache in memory 713 may store calculated changes to the profit per square foot based on modifications to product displays.

Processing device 712 may perform computation and control functions of a system and comprises a suitable central processing unit (CPU). Processing device 712 may include a single integrated circuit, such as a microprocessing device, or may include any suitable number of integrated circuit devices and/or circuit boards working in cooperation to accomplish the functions of a processing device. Processing device 712 may execute computer programs, such as object-oriented computer programs, within memory 713.

The foregoing description has been presented for purposes of illustration and description. It is not exhaustive and does not limit embodiments of the disclosure to the precise forms disclosed. For example, although the processing device 712 is shown as separate from the modules 714 and 715 and the touch screen interface 711, in some instances the processing device 712 and the touch screen interface 711 and/or one or more of the modules 714 and 715 may be functionally integrated to perform their respective functions.

It will be apparent to those skilled in the art that various modifications and variations can be made in the VR/AR phobia training in a controlled environment with stress level sensors and management through scenarios control of the present invention without departing from the spirit or scope of the invention. Thus, it is intended that the present invention cover the modifications and variations of this invention provided they come within the scope of the appended claims and their equivalents.

What is claimed is:

1. A method comprising:
selecting a phobia simulation;
navigating a phobia simulation using augment reality or virtual reality;
collecting one or more biometrics of a patient during the phobia simulation; and
determining a stress level of the patient.

2. The method according to claim 1, wherein the simulated phobia includes a video stream displayed on augmented or virtual reality glasses.

3. The method according to claim 1, wherein the one or more biometrics are selected from heart rate or blood pressure.

4. The method according to claim 1, wherein the one or more biometrics are selected from blood oxygen or finger temperature.

5. The method according to claim 1, wherein the one or more biometrics are selected from heart rate variability, electrocardiogram (ECG), electroencephalogram (EEG).

6. The method according to claim 1, wherein the phobia simulation includes haptic feedback.

7. The method according to claim 1, wherein a healthcare professional monitors the biometrics of the patient in real-time.

8. A non-transitory computer readable storage medium storing one or more programs configured to be executed by a processor, the one or more programs comprising instructions for:
selecting a phobia simulation;
navigating a phobia simulation using augment reality or virtual reality;
collecting one or more biometrics of a patient during the phobia simulation; and
determining a stress level of the patient.

9. The computer readable storage medium of claim 8, wherein the simulated phobia includes a video stream displayed on augmented or virtual reality glasses.

10. The computer readable storage medium of claim 8, wherein the one or more biometrics are selected from heart rate or blood pressure.

11. The computer readable storage medium of claim 8, wherein the one or more biometrics are selected from blood oxygen or finger temperature.

12. The computer readable storage medium of claim 8, wherein the one or more biometrics are selected from heart rate variability, electrocardiogram (ECG), electroencephalogram (EEG).

13. The computer readable storage medium of claim 8, wherein the phobia simulation includes haptic feedback.

14. The computer readable storage medium of claim 8, wherein a healthcare professional monitors the biometrics of the patient in real-time.

15. A portable electronic device comprising:
one or more processors; and
a memory storing one or more programs for execution by the one or more processors, the one or more programs including instructions for:
selecting a phobia simulation;
navigating a phobia simulation using augment reality or virtual reality;
collecting one or more biometrics of a patient during the phobia simulation; and
determining a stress level of the patient.

16. The portable electronic device according to claim 15, wherein the simulated phobia includes a video stream displayed on augmented or virtual reality glasses.

17. The portable electronic device according to claim 15, wherein the one or more biometrics are selected from heart rate or blood pressure.

18. The portable electronic device according to claim 15, wherein the one or more biometrics are selected from heart rate variability, electrocardiogram (ECG), electroencephalogram (EEG).

19. The portable electronic device according to claim 15, wherein the phobia simulation includes haptic feedback.

20. The portable electronic device according to claim 15, wherein a healthcare professional monitors the biometrics of the patient in real time.
